(19) United States
(12) Patent Application Publication
(10) Pub. No.: US 2002/0082200 A1
(43) Pub. Date: Jun. 27, 2002

(54) Use of inhibitors of human S-CD23

(75) Inventors: Gary Christie, Bishop's Stortford (GB); Beverly Jane Weston, Peaslake (GB)

Correspondence Address: GLAXOSMITHKLINE, Corporate Intellectual Property - UW2220, P.O.Box 1539, King of Prussia, PA, 19406-0939, US

(73) Assignee: SmithKline Beecham p.1.c.

(21) Appl. No.: 10/038,331

(22) Filed: Jan. 3, 2002

Related U.S. Application Data
(63) Continuation of application No. 09/852,807, filed on May 10, 2001.
Application No. 09/852,807 is a continuation of application No. 09/266,050, filed on Mar. 10, 1999, now abandoned.
Application No. 09/266,050 is a continuation-in-part of application No. 09/101,821, filed on Sep. 16, 1998, now abandoned.
Application No. 09/266,050 is a continuation-in-part of application No. 08/776,133, filed on Aug. 11, 1997, now abandoned.

(30) Foreign Application Priority Data
Jul. 13, 1994 (GB) 9414157.9
Jan. 17, 1996 (GB) 9601042.6

(51) Int. Cl. A61K 38/05; A61K 31/405; A61K 31/24; A61K 31/165

(52) U.S. Cl. 514/7; 514/19; 514/419; 514/575; 514/534; 514/620

(57) ABSTRACT

Inhibitors of matrix metalloproteases such as collagenase are capable of inhibiting the release of human soluble CD23 and are therefore useful in the treatment and prophylaxis of conditions in which an excess of s-CD23 is implicated, such as allergy and autoimmune disease.

This invention relates to a medical use and in particular to the use of inhibitors of the formation of soluble human CD23 for the treatment of conditions associated with excess production of soluble CD23 (s-CD23) such as autoimmune disease and allergy.

Matrix metalloproteases such as collagenase, stromelysin and gelatinase are known to be involved in connective tissue breakdown. Known classes of matrix metalloprotease inhibitors include derivatives of hydroxamic acid, phosphonates and thiols.

International Patent Application, Publication Number WO 93/20047 discloses that inhibitors of the matrix metalloproteases, especially derivatives of hydroxamic acid, are potentially useful for the treatment or prophylaxis of conditions involving such tissue breakdown, for example rheumatoid arthritis, osteoarthritis, osteopenias such as osteoporosis, periodontitis, gingivitis, corneal epidermal or gastric ulceration, and tumour metastasis or invasion.

WO 93/20047 discloses various derivatives of hydroxamic acid including those from the following patent publications: U.S. Pat. No. 4,599,361, EP-A-0236872, EP-A-0274453, WO 90/05716, WO 90/05719, WO 91/02716, EP-A-0489577, EP-A-0489579, EP-A-0497192 and WO 92/13831.

CD23 (the low affinity IgE receptor FcεRII, Blast 2), is a 45 kDa type II integral protein expressed on the surface of a variety of mature cells, including B and T lymphocytes, macrophages, natural killer cells, Langerhans cells, monocytes and platelets (Delespesse et al, Adv Immunol, 49 [1991] 149-191). There is also a CD23-like molecule on eosinophils (Grangette et al, J Immunol, 143 [1989] 3580-3588). CD23 has been implicated in the regulation of the immune response (Delespesse et al, Immunol Rev, 125 [1992] 77-97). Human CD23 exists as two differentially regulated isoforms, a and b, which differ only in the amino acids at the intracellular N-terminus (Yokota et al, Cell, 55 [1988] 611-618). In man the a isoform is found only on B-lymphocytes, whereas type b is found on all other cells capable of expressing CD23. However, expression of the b isoform on B-lymphocytes is inducible by IL4. Intact, cell bound CD23 (i-CD23) is known to undergo cleavage from the cell surface leading to the formation of a number of well-defined soluble fragments (s-CD23), which are produced as a result of a complex sequence of proteolytic events, the mechanism of which is still poorly understood (Bourget et al J Biol Chem, 269 [1994] 6927-6930). Although not yet proven, it is postulated that the major soluble fragments (Mr 37, 33, 29 and 25 kDa) of these proteolytic events, all of which retain the C-terminal lectin domain common to i-CD23, occur sequentially via initial formation of the 37 kDa fragment (Letellier et al, J Exp Med, 172 [1990] 693-700). An alternative intracellular cleavage pathway leads to a stable 16 kDa fragment differing in the C-terminal domain from i-CD23 (Grenier-Brosette et al, Eur J Immunol, 22 [1992] 1573-1577).

Several activities have been ascribed to membrane bound i-CD23 in humans, all of which have been shown to play a role in IgE regulation. Particular activities include: a) antigen presentation, b) IgE mediated eosinophil cytotoxicity, c) B cell homing to germinal centres of lymph nodes and spleen, and d) downregulation of IgE synthesis (Delespesse et al, Adv Immunol, 49, [1991] 149-191). The three higher molecular weight soluble CD23 fragments (Mr 37, 33 and 29 kDa) have multifunctional cytokine properties which appear to play a major role in IgE production. Thus, the excessive formation of s-CD23 has been implicated in the overproduction of IgE, the hallmark of allergic diseases such as extrinsic asthma, rhinitis, allergic conjuctivitis, eczema, atopic dermatitis and anaphylaxis (Sutton and Gould, Nature, 366, [1993] 421-428). Other biological activities attributed to s-CD23 include the stimulation of B cell growth and the induction of the release of mediators from monocytes. Thus, elevated levels of s-CD23 have been observed in the serum of patients having B-chronic lymphocytic leukaemia (Sarfati et al, Blood, 71 [1988] 94-98) and in the synovial fluids of patients with rheumatoid arthritis (Chomarat et al, Arthritis and Rheumatism, 36 [1993] 234-242).

Because of these various properties of CD23, compounds which inhibit the formation of s-CD23 should have twofold actions of a) enhancing negative feedback inhibition of IgE synthesis by maintaining levels of i-CD23 on the surface of B cells, and b) inhibiting the immunostimulatory cytokine activities of higher molecular weight soluble fragments (Mr 37, 33 and 29 kDa) of s-CD23.

It has now surprisingly been found that compounds which inhibit the action of matrix metalloproteases (eg collagenase, stromelysin and gelatinase) are effective inhibitors of the release of human soluble CD23 transfected into mammalian cell culture systems. It is also indicated that such compounds inhibit the formation of IgE by human peripheral blood mononuclear cells in response to IL4 and stimulation with an antibody to CD40. Inhibitors of the matrix metalloproteases are therefore potentially useful for the treatment or prophylaxis of disorders such as allergy and autoimmune disease in which the overproduction of s-CD23 is implicated. Known classes of matrix metalloprotease inhibitors include derivatives of hydroxamic acid, phosphonic acid and thiols, all of which have been shown to inhibit CD23 proteolysis.

Accordingly, the present invention provides the use of an inhibitor of the formation of human soluble CD23, such as an inhibitor of matrix metalloproteases, for the production of a medicament for the treatment or prophylaxis of disorders such as allergy and autoimmune disease in which the overproduction of s-CD23 is implicated.

In a further aspect the invention provides a method for the treatment or prophylaxis of disorders such as allergy and autoimmune disease in which the overproduction of s-CD23 is implicated, which method comprises the administration of an inhibitor of the formation of soluble human CD23, such as an inhibitor of matrix metalloproteases, to a human or non-human mammal in need thereof.

The invention also provides a pharmaceutical composition for the treatment or prophylaxis of disorders such as allergy and autoimmune disease in which the overproduction of s-CD23 is implicated which comprises an inhibitor of the formation of soluble human CD23, such as an inhibitor of matrix metalloproteases and optionally a pharmaceutically acceptable carrier therefor.

Suitable matrix metalloprotease inhibitors are set out in the Table and include the hydroxamic acid derivatives disclosed in WO 90/05716, WO 90/05719, WO 91/02716, WO 92/13831, WO 93/20047, EP-A-0236872, EP-A-0274453, EP-A-0489577, EP-A-0489579, EP-A-0497192 and U.S. Pat. No. 4,599,361.

Suitable matrix metalloprotease inhibitors also include the thiols and phosphonic acids disclosed in EP 273689 and EP320118.

The contents of WO 90/05716, WO 90/05719, WO 91/02716, WO 92/13831, WO 93/20047, EP-A-0236872, EP-A-0274453, EP-A-0489577, EP-A-0489579, EP-A-0497192, U.S. Pat. No. 4,599,361, EP 273689 and EP320118, and the other patent publications referred to in the Table, are incorporated herein by reference, including the specific examples disclosed in these patent publications.

Particular matrix metalloprotease inhibitors include the compounds disclosed hereinafter in the Procedures section.

Favoured matrix metalloprotease inhibitors include Example 2 of WO 90/05719 and Example 1 of EP 0497192.

It is to be understood that the pharmaceutically acceptable salts, solvates and other pharmaceutically acceptable derivatives of the above mentioned matrix metalloproteases inhibitors are also included in the present invention.

The matrix metalloprotease inhibitors mentioned herein may exist in several different isomeric forms, including stereoisomeric forms. Unless specifically stated to the contrary herein with respect to particular compounds, all isomers including stereoisomers and mixtures of isomers, such as racemic mixtures, are included within the present invention.

The matrix metalloprotease inhibitors of the invention may be prepared by use of any appropriate conventional method, for example the matrix metalloprotease inhibitors disclosed in patent publications WO 90/05716, WO 90/05719, WO 91/02716, WO 92/13831, WO 93/20047, EP-A-0236872, EP-A-0274453, EP-A-0489577, EP-A-0489579, EP-A-0497192 and U.S. Pat. No. 4,599,361, EP 273689 and EP320118 may be prepared by the methods disclosed therein.

The isomers, including stereoisomers, of the matrix metalloprotease inhibitors of the present invention may be prepared as mixtures of such isomers or as individual isomers. The individual isomers may be prepared by any appropriate method, for example individual stereoisomers may be prepared by stereospecific chemical synthesis starting from chiral substrates or by separating mixtures of enantiomers using known methods.

It is preferred that the matrix metalloprotease inhibitors are isolated in substantially pure form.

As used herein the term “matrix metalloprotease inhibitor” and equivalent terms means any compound which inhibits any member of the family of zinc and calcium dependent endopeptidases (matrix metalloproteases) that have the ability to degrade components of the connective tissue matrices. Matrix metalloproteases and their inhibition are discussed by inter alia Hooper, FEBS Letters 1994, 354,1-6; Gordon et al., Clinical and Experimental Rheumatology 1993, 11 (Suppl. 8), S91-S94; Woessner, FASEB 1991, 5, 2145-2154; and Birkedal-Hansen, Critical Reviews in Oral Biology and Medicine 1993, 4(2), 197-250. Assays for inhibition of collagenase, stromelysin, and gelatinase are described in WO 90/05719, page 67, WO 90/05719, page 68, and EP-A-0 489 577, pages 25-26, respectively. The present invention comprehends the use of compounds which are deemed active in any one of these assays, as well as the specific compounds set out in the Table.

As stated herein an inhibitor of the formation of soluble human CD23, such as a matrix metalloprotease inhibitor, has useful medical properties. Preferably the active compounds are administered as pharmaceutically acceptable compositions.

The compositions are preferably adapted for oral administration. However, they may be adapted for other modes of administration, for example in the form of a spray, aerosol or other conventional method for inhalation, for treating respiratory tract disorders; or parenteral administration for patients suffering from heart failure. Other alternative modes of administration include sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions of this invention may also suitably be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns for example diameters in the range of 1-50 microns, 1-10 microns or 1-5 microns. Where appropriate, small amounts of other anti-asthmatics and bronchodilators, for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included.

The compositions may contain from 0.1% to 99% by weight, preferably from 10-60% by weight, of the active material, depending upon the method of administration. A preferred range for inhaled administration is 10-99%, especially 60-99%, for example 90, 95 or 99%.

Microfine powder formulations may suitably be administered in an aerosol as a metered dose or by means of a suitable breath-activated device.

Suitable metered dose aerosol formulations comprise conventional propellants, cosolvents, such as ethanol, surfactants such as oleyl alcohol, lubricants such as oleyl alcohol, desiccants such as calcium sulphate and density modifiers such as sodium chloride.

Suitable solutions for a nebulizer are isotonic sterilised solutions, optionally buffered, at for example between pH 4-7, containing up to 20 mg/ml of compound but more generally 0.1 to 10 mg/ml, for use with standard nebulisation equipment.

An effective amount will depend on the relative efficacy of the compounds of the present invention, the severity of the disorder being treated and the weight of the sufferer. Suitably, a unit dose form of a composition of the invention may contain from 0.1 to 1000 mg of a compound of the invention (0.001 to 10 mg via inhalation) and more usually from 1 to 500 mg, for example 1 to 25 or 5 to 500 mg. Such compositions may be administered from 1 to 6 times a day, more usually from 2 to 4 times a day, in a manner such that the daily dose is from 1 mg to 1 g for a 70 kg human adult and more particularly from 5 to 500 mg. That is in the range of about 1.4×10⁻² mg/kg/day to 14 mg/kg/day and more particularly in the range of about 7×10⁻² mg/kg/day to 7 mg/kg/day.

| | | TABLE |
|---|---|---|
| | | Specific compounds |
| | | and methods of |
| | | preparation-Example |
| Patent publication | Compounds disclosed | Nos. |
| US-A-4,595,700 | Compounds of formula | 1 to 8. |
| US-A-4,599,361 | (I) as defined in claim 1, | 1 to 7. |
| GB-A-2 268 934 | optionally as further | 1 to 10. |
| GB-A-2 272 441 | subdefined in the | 1 to 5. |
| EP-A-0 231 081 | description. | 1 to 8. |
| EP-A-0 236 872 | | 1 to 28. |
| EP-A-0 262 053 | | 1 to 15. |
| EP-A-0 273 689 | | 1 to 38. |
| EP-A-0 276 436 | | 1 to 44. |
| EP-A-0 274 453 | | 1 to 8. |
| EP-A-0 320 118 | | 1 to 5. |
| EP-A-0 489 577 | | 1 to 25. |
| EP-A-0 489 579 | | 1 to 4. |
| EP-A-0 497 192 | | 1 to 80. |
| EP-A-0 498 665 | | 1 to 27. |
| EP-A-0 520 573 | | 1 to 34. |
| EP-A-0 574 758 | | 1 to 43. |
| EP-A-0 575 844 | | 1 to 27. |
| EP-A-0 606 046 | | 1 to 32. |
| EP-A-0 613 883 | | 1 to 7. |
| EP-A-0 621 270 | | 1 to 40. |
| WO 90/05716 | | 1 to 38. |
| WO 90/05719 | | 1 to 26. |
| WO 91/02716 | | 1 to 17. |
| WO 92/09563 | Compounds of formula | 1 to 21. |
| | (1) or (2) as defined in | |
| | claim 1, optionally as | |
| | further subdefined in the | |
| | description. | |
| WO 92/13831 | Compounds of formula | 1 to 27. |
| WO 92/21360 | (I) as defined in claim 1, | 1 to 5. |
| WO 92/22523 | optionally as further | I to X. |
| WO 93/14096 | subdefined in the | 1 to 8. |
| WO 93/20047 | description. | 1 to 14. |
| WO 93/24475 | | 1 to 6. |
| WO 93/24449 | | 1 to 8. |
| WO 94/00119 | | 1 to 86. |
| WO 94/07481 | | 1 to 15. |
| WO 94/12169 | | 1 to 24. |
| WO 94/21625 | | 1 to 7. |
| WO 94/21612 | | 1 to 116. |
| WO 94/24140 | | 1 to 5. |
| WO 94/25434 | | 1 to 7. |
| WO 94/25435 | | Example 1. |
| WO 95/04033 | | Examples 1 to 7. |
| WO 95/04715 | | All examples. |
| WO95/09833 | | All |
| WO 95/12603 | | All |
| WO95/13289 | | All |
| WO95/19956 | | All |
| WO95/19961 | | All |
| WO95/22966 | | All |
| WO95/23790 | | All |
| WO95/29689 | | All |
| WO95/29892 | | All |
| EP-A-0 684240 | | All |
| JPO7196598 | | All |
| WO95/19957 | Specific compounds of | All |
| | Claim 1 or any other | |
| | claim. | |

The following examples illustrate the invention but do not limit it in any way.

Preparations:

Preparation 1: [4-(N-Hydroxyamino)-2-(R)-isobutyl-3-(S)-(2-thiophenethiomethyl)succinyl]-(S)-phenylalanine-N-methylamide, Sodium Salt

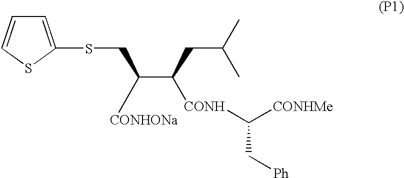

This is prepared according to the procedure disclosed in WO 90/05719 (see example 11, the free acid being prepared in example 2).

Preparation 2: N²-[(R)-[Hydroxycarbamoylmethyl]-4-methylvaleryl]-N¹, 3-dimethyl-(S)-valinamide.

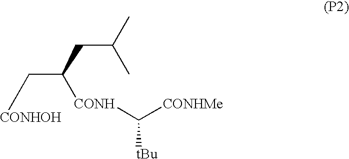

This is prepared according to the procedure disclosed in EP 0497192 (see example 1).

Preparation 3: N-[3-(N′-Hydroxycarboxamido)-2-(2-methylpropyl)-propanoyl]-(S)-O-methyl-L-tyrosine-N-methylamide

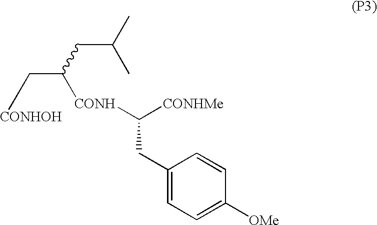

This is prepared according to the procedure disclosed in U.S. Pat. No. 4,599,361 (see example 1).

Preparation 4: Methyl 3-(S)-mercapto-6-methyl-4-(S)-[[[1(S)-[(methylamino)carbonyl]-2-(3-indolyl)ethyl]amino]carbonyl]heptanoate

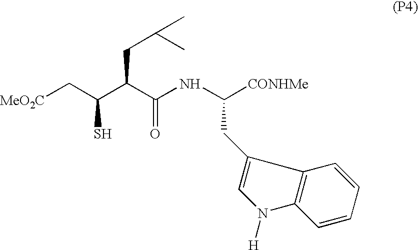

This is prepared according to the procedure disclosed in EP 273689 and J. Medicinal Chemistry 1993, 36, 4030-40 (see compound 56).

Preparation 5: Isopropyl 3-(S)-mercapto-6-methyl-4-(S)-[[[1(S)-[(methylamino)carbonyl]-2-(3-indolyl)ethyl]amino]carbonyl]heptanoate

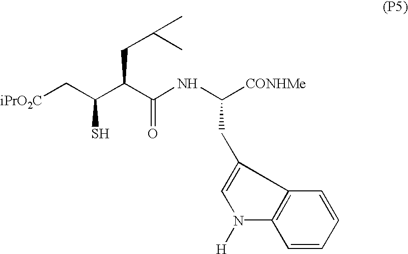

This is prepared according to the procedures disclosed in EP 273689.

Preparation 6: 3-(S)-Mercapto-N¹-[1-(S)-[(methylamino)carbonyl]-2-(4-methoxyphenyl)ethyl]-2-(S)-(2-methylpropyl)pentanediamide

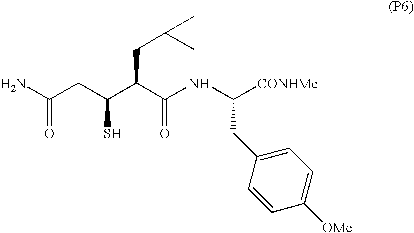

This is prepared according to the procedure disclosed in J. Medicinal Chemistry ibidem (see compound 47a).

Preparation 7: N-[N-((S)-1-Phosphonopropyl)-(S)-leucyl]-O-methyl-(S)-tyrosine N-methylamide

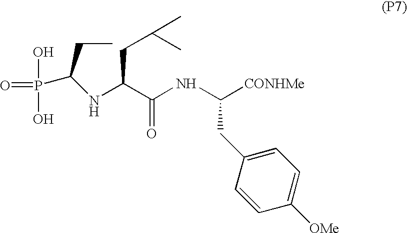

This is prepared according to the procedure disclosed in EP 320118 and J. Medicinal Chemistry 1994, 37, 158-169 (see compound 12).

Preparation 8: N-[3-(Hydroxycarboxamido)-2R-(2-methylpropyl)propanoyl]-(S)-phenylalanine-N-methylamide

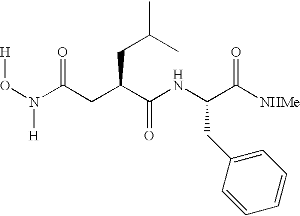

This is prepared by hydrogenolysis (using Pd/BaSO₄ as catalyst) of the precursor benzhydroxamate, itself prepared from the analogous carboxylic acid and O-benzylhydroxylamine using similar methodology to that described in WO 90/05719 example 1g

Preparation 9: N-[3-(Hydroxycarboxamido)-2R-(2-methylpropyl)propanoyl]-(S)-phenylalanine-N-benzylamide

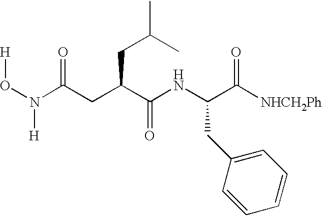

This is prepared from the precursor carboxylic acid and O-trimethylsilylhydroxylamine using similar methodology to that described in WO 90/05719 example 1g but with bromo-tris-pyrrolidino-phosphonium hexafluorophosphate replacing water soluble carbodiimide as coupling agent.

Biological Test Methods

Procedure 1:

The ability of test compounds to inhibit the release of soluble CD23 was investigated by use of the following procedure.

Adherent Chinese Hamster Ovary cells which had been transfected with the alpha form of CD23 were grown in microtitre plates. Cells were grown to confluence in α-MEM medium with 10% foetal calf serum, 2 mM glutamine containing 800 micro g/ml G418. Medium was removed and the cells washed with sterile phosphate buffered saline. Test compounds were dissolved in dimethyl sulphoxide at a stock concentration of 20 mM, then diluted 1 in 200 with α-MEM containing 800 micro g/ml G418 (no foetal calf serum). 100 ml of the diluted compounds were added to the adherent cells in triplicate wells. Appropriate control cultures were set up in triplicate. The plates were incubated for 6 hours at 37° C., 95% air/5% CO₂ in a humidified incubator, then centrifuged at 200× g for 3 minutes. A specific ELISA for CD23, obtained from The Binding Site Limited, Institute of Research and Development, Birmingham England, was used to measure CD23 levels in the culture supernatants.

The average concentration (IC₅₀) of test compound which inhibits the release of soluble CD23 by 50% relative to the control culture was determined.

Results

| Test Compound | |
|---|---|
| No. | IC₅₀ (microM) |
| P1 | 0.05 |
| P2 | 0.05 |
| P3 | 3.35 |
| P4 | 2.2 |
| P5 | 60 |
| P6 | 60 |
| P7 | 30 |

Procedure 2:

The ability of test compounds to inhibit the release of soluble CD23 was also investigated by use of the following procedure.

RPMI 8866 Cell membrane CD23 Cleavage Activity Assay:

Plasma membranes from RPMI 8866 cells, a human Epstein-Barr virus transformed B-cell line (Sarfati et al., Immunology 60 [1987] 539-547) expressing high levels of CD23 are purified using an aqueous extraction method. Cells resuspended in homogenization buffer (20 mM HEPES pH 7.4, 150 mM NaCl, 1.5 mM MgCl2, 1 mM DTT) are broken by N2 cavitation in a Parr bomb and the plasma membrane fraction mixed with other membranes is recovered by centrifugation at 10,000× g. The light pellet is resuspended in 0.2 M potassium phosphate, pH 7.2 using 2 ml per 1-3 g wet cells and the nuclear pellet is discarded. The membranes are further fractionated by partitioning between Dextran 500 (6.4% w/w) and polyethylene glycol (PEG) 5000 (6.4% w/w) (ref), at 0.25 M sucrose in a total of 16 g per 10-15 mg membrane proteins [Morre and Morre, BioTechniques 7, 946-957 (1989)]. The phases are separated by brief centrifugation at 1000× g and the PEG (upper) phase is collected, diluted 3-5 fold with 20 mM potassium phosphate buffer pH 7.4, and centrifuged at 100,000× g to recover membranes in that phase. The pellet is resuspended in phosphate-buffered saline and consists of 3-4 fold enriched plasma membranes as well as some other cell membranes (e.g. lysosomes, Golgi). The membranes are aliquoted and stored at −80° C. Fractionation at 6.6% Dextran/PEG yields plasma membranes enriched 10-fold.

The fractionated membranes are incubated at 37° C. for times up to 4 hrs to produce fragments of CD23 which are separated from the membrane by filtration in 0.2 micron Durapore filter plates (Millipore) after quenching the assay with 5 uM Preparation 1 from P 30994. sCD23 released from the membrane is determined using the EIA kit from The Binding Site (Birmingham, UK) or a similar one utilizing MHM6 anti-CD23 mAb [Rowe et al., Int. J. Cancer, 29, 373-382 (1982)] or another anti-CD23 mAb as the capture antibody in a sandwich EIA. The amount of soluble CD23 made by 0.5 ug membrane protein in a total volume of 50 ul phosphate-buffered saline is measured by EIA and compared to the amount made in the presence of various concentrations of inhibitors. Inhibitors are prepared in solutions of water or dimethylsulfoxide (DMSO) and the final DMSO concentration is not more than 2%. IC50's are determined by curve fitting as the concentration where 50% inhibition of production of sCD23 is observed relative to the difference in sCD23 between controls incubated without inhibitor.

Procedure 3:

The ability of test compounds to inhibit the formation of human IgE in vitro was investigated using the following procedure:

Human peripheral blood mononuclear cells were separated by centrifugation over Ficoll-Paque (Pharmacia). The cells were suspended in RPMI 1640 medium containing 10% foetal calf serum, 2 mM glutamine, 50 microM 2-mercaptoethanol and 50 micro g/ml gentamycin (TCM) at a concentration of 1.25×10⁶ cells/ml. 800 micro l of the cell suspension were aliquoted into the wells of a 48 well plate. 100 micro l of TCM or IL4 at 500 ng/ml was added in quadriplicate to the appropriate wells, followed by 100 micro l of TCM or 10× the final concentration of compound under investigation. Test compounds are dissolved in dimethylsulphoxide (DMSO) at a stock dilution of

10⁻²M diluted 1 in 100 in TCM and then as above. The plates are incubated for 12 days at 37° C. in a 95% air/5% CO₂ humidified incubator. At the end of the culture period the supernatants were removed with the wells and centrifuged (200× g for 10 minutes) to remove any non-adherent cells. There was no toxicity as assessed by trypan blue dye exclusion. The IgE concentration in the supernatants was measured by ELISA.

Results

| | | IgE ng/ml (mean +/− sem) |
|---|---|---|
| TCM | | 0.25 +/− 0.08 |
| IL4 (5 ng/ml) | | 72.4 |
| IL4 with P1: | 10⁻⁵ M | 3.2 +/− 1.9 |
| | 10⁻⁶ M | 21.3 +/− 16.5 |
| | 10⁻⁷ M | 68.8 +/− 21.8 |

Procedure 4:

The ability of test compounds to inhibit the formation of human IgE in vitro was investigated using the following procedure:

Human tonsillar B lymphocytes were suspended in RPMI 1640 medium containing 10% foetal calf serum, 2 mM glutamine, 50 micro M 2-mercaptoethanol and 50 micro g/ml gentamycin (TCM) at a concentration of 1.25×10⁶ cells/ml. 800 micro l of the cell suspension were aliquoted into the wells of a 48 well plate. 100 micro l of TCM or IL4 at 100 ng/ml and antibody to CD40 at 10 microg/ml was added in quadriplicate to the appropriate wells, followed by 100 micro l of TCM or 10× the final concentration of compound under investigation. Test compounds are dissolved in DMSO at a stock dilution of 10⁻²M diluted 1 in 100 in TCM and then as above. The plates are incubated for 11 days at 37° C. in a 95% air/5% CO₂ humidified incubator. At the end of the culture period the supernatents were removed from the wells and centrifuged (200× g for 10 minutes) to remove any non-adherent cells. There was no toxicity as assessed by trypan dye exclusion. The IgE concentration in the supernatants was measured by ELISA.

Results

| | | IgE ng/ml (mean +/− sem) |
|---|---|---|
| TCM | | 1.9 |
| IL4 (10 ng/ml) and anti CD40 (1 micro g/ml) | | 11.7 +/− 1.1 |
| IL4 with P1: | 10⁻⁵ M | 2.9 +/− 0.2 |
| | 10⁻⁶ M | 4.3 +/− 0.6 |
| | 10⁻⁷ M | 6.2 +/− 0.5 |

1. A method for the treatment or prophylaxis of disorders in which the overproduction of s-CD23 is implicated, which method comprises the administration of an effective amount of an inhibitor of the formation of human soluble CD23 to a human or non-human mammal in need thereof, with the provisos that:
(a) the disorder is not mediated by a matrix metalloprotease or by tissue necrosis factor; and
(b) the inhibitor does not form part of the state of the art by virtue of WO92/16517 or WO93/18173.

2. The method according to claim 1, wherein the inhibitor of the formation of s-CD23 is an inhibitor of matrix metalloprotease.

3. The method according to claim 1, wherein the inhibitor of the formation of s-CD23 is a hydroxamic acid derivative, a phosphate or a thiol.

4. The method according to claim 1, wherein the inhibitor of the formation of s-CD23 is selected from:
[4-(N-hydroxyamino)-2-(R)-isobutyl-3-(S)-(2-thiophenethiomethyl)succinyl]-(S)-phenylalanine-N-methylamide;
N²-[(R)-[hydroxycarbamoylmethyl]-4-methylvaleryl]-N¹, 3-dimethyl-(S)-valinamide;
N-[3-(N′-hydroxycarboxamido)-2-(2-methylpropyl)propanoyl]-(S)-O-methyl-L-tyrosine-N-methylamide;
methyl 3-(S)-mercapto-6-methyl-4-(S)-[[[1(S)-[(methylamino)carbonyl]-2-(3-indolyl)ethyl]amino]carbonyl]heptanoate;
isopropyl 3-(S)-mercapto-6-methyl-4-(S)-[[[1(S)-[(methylamino)carbonyl]-2-(3-indolyl)ethyl]amino]carbonyl]heptanoate;
3-(S)-mercapto-N¹-[1-(S)-[(methylamino)carbonyl]-2-(4-methoxyphenyl)ethyl]-2-(S)-(2-methylpropyl)pentanediamide;
N-[N-((S)-1-phosphonopropyl)-(S)-leucyl]-O-methyl-(S)-tyrosine N-methylamide;
N-[3-(hydroxycarboxamido)-2R-(2-methylpropyl)propanoyl]-(S)-phenylalanine-N-methylamide; and
N-[3-(hydroxycarboxamido)-2R-(2-methylpropyl)propanoyl]-(S)-phenylalanine-N-benzylamide; or
a pharmaceutically acceptable salts thereof.

5. The method according to claim 1, wherein the inhibitor is hereinbefore described with reference to the Table.
